(19) United States
(12) Patent Application Publication
(10) Pub. No.: US 2017/0320951 A1
(43) Pub. Date: Nov. 9, 2017

(54) METHODS FOR DIAGNOSING AND TREATING CD1D RESTRICTED GAMMA/DELTA T CELL LYMPHOMAS

(71) Applicants: Institut National De La Santé Et De La Recherche Médicale (INSERM), Paris (FR); Université Claude Bernard Lyon 1, Villeurbanne (FR); Ecole Normale Supérieure De Lyon, Lyon (FR); Centre National De La Recherche Scientifique (CNRS), Paris (FR)

(72) Inventors: Laurent Genestier, Lyon (FR); Emmanuel Bachy, Pierre Benite (FR); Thierry Defrance, Lyon (FR)

(73) Assignees: Institut National De La Santé Et De La Recherche Médicale (INSERM), Paris (FR); Université Claude Bernard Lyon 1, Villeurbanne (FR); Ecole Normale Supérieure De Lyon, Lyon (FR); Centre National De La Recherche Scientifique (CNRS), Paris (FR)

(21) Appl. No.: 15/412,452

(22) Filed: Jan. 23, 2017

Related U.S. Application Data
(63) Continuation of application No. PCT/EP2015/064933, filed on Jul. 1, 2015.

(30) Foreign Application Priority Data
Jul. 2, 2014 (EP) 14306073.9

(51) Int. Cl. C07K 16/28; A61K 39/395; C07K 16/30; G01N 33/574; C12N 15/115; A61K 38/13; A61K 31/713; A61K 39/00

(52) U.S. Cl. CPC C07K 16/2833; A61K 38/13; A61K 39/39558; A61K 31/713; C07K 16/3061; G01N 33/57407; C12N 15/115; A61K 2039/505; C07K 2317/21; C07K 2317/24; C07K 2317/76; C12N 2310/16; C12N 2320/31; G01N 2333/70539; C07K 2317/569

(57) ABSTRACT

Methods for diagnosing and treating CD1d-restricted gamma/delta T cell lymphomas are disclosed. In particular, the present disclosure relates to a method for diagnosing a T cell lymphoma as a CD1d-restricted gamma/delta T cell lymphoma in a patient in need thereof including i) detecting the presence of CD1d restricted gamma/delta T lymphoma cells in a cell lymphoma sample obtained from the patient and ii) concluding that the T cell lymphoma is a CD1d-restricted gamma/delta T cell lymphoma when the presence of CD1d restricted gamma/delta T cells is detected in the sample. The present disclosure also relates to a method for treating a CD1d-restricted gamma/delta T cell lymphoma as diagnosed by the diagnostic method, including administering the patient with a therapeutically effective amount of a CD1d antagonist.

### CROSS-REFERENCE TO RELATED APPLICATIONS

This application is a continuation of International Patent Application Serial No. PCT/EP2015/064933, filed on Jul. 1, 2015, which claims priority to European Patent Office Serial Number 14306073.9, filed on Jul. 2, 2014, both of which are incorporated by reference herein.

### TECHNICAL FIELD

The present invention relates to methods for diagnosing and treating CD1d restricted gamma/delta T cell lymphomas.

### BACKGROUND

Identifying the cellular lineage of lymphomas is a field of intense research and has been fruitfully applied to B-cell lymphoma classification. Hence, unraveling the correlations between B-cell lymphoma subtypes and normal B-cell development helped understanding transformation mechanisms and formed the basis for the current classification in humans as well as for differential therapies. Such a correspondence between normal development stages and the initiating cell in T-cell lymphoma is still lacking. Except for angio-immunoblastic T-cell lymphoma (AITL) whose normal counterpart was identified as follicular helper T cells (T_{FH}), the normal cellular derivation of most mature T-cell malignancies is still merely speculative. The complexity of the T-cell adaptative immune system encompassing numerous subsets with effector, memory and regulatory functions might explain why peripheral T cell lymphomas (PTCL) still remain poorly defined.

There are 2 mutually exclusive subtypes of CD3-associated T-cell receptor (TCR) molecules expressed on normal T cells, namely alpha/beta and gamma/delta. In contrast with the alpha/beta T cells, which are mostly CD4+ or CD8+, the gamma/delta T cells are primarily CD4⁻ and CD8⁻. These cells frequently express natural killer (NK) cell-associated antigens (CD16, CD56) and have cytotoxic activity. Although the exact pathophysiologic role of gamma/delta T cells is unknown, reports suggest that that these cells may have a role in the immune reaction during infection and in the regulation of pathophysiologic autoimmune responses. Like their normal counterparts in the peripheral blood (PB), most of the peripheral (postthymic) T-cell lymphomas (PTCLs) are also of alpha/beta T-cell derivation, characterized as CD2+, CD3+, and CD4+ or CD8+. They commonly have prominent bone marrow (BM) involvement. PTCLs originating from gamma/delta T cells are rare and most show a homing pattern reminiscent of normal gamma/delta T cells, which preferentially occupy the sinusoidal areas of the spleen, intestinal mucosa, and skin. Their phenotype is typically CD2+, CD3+, CD4⁻, and CD8⁻. The very low incidence of PTCLs originating from gamma/delta T cell, along with its propensity to mimic different pathological entities, makes this type lymphoma a true diagnostic challenge. For all these reasons, gamma/delta T cell lymphomas represent an interesting field of investigation aimed at improving diagnostic and therapeutic performances.

### SUMMARY

The present invention relates to methods for diagnosing and treating CD1d-restricted gamma/delta T cell lymphomas. In particular the present invention is defined by the claims.

### BRIEF DESCRIPTION OF THE DRAWINGS

The invention will be further illustrated by the following figures and examples. However, these examples and figures should not be interpreted in any way as limiting the scope of the present invention.

FIGS. 1A and 1B are a series of graphs showing an identification of CD1d sulfatide-reactive cells in V81 but not V82 lymphomas. FIG. 1A shows a flow cytometry analysis of HSTL and PTCL-NOS patients showing V81 or V82 versus no tetramer or human CD1d tetramer (loaded with aGalCer, sulfatide or unloaded) stainings. FIG. 1B shows human CD1d tetramer (loaded with aGalCer, sulfatide or unloaded) stainings on lymphoma cells gated on V81 or V82⁺ cells.

FIG. 2 is a series of graphs showing that PTCL rely on TCR/CD1d interaction for engraftment and survival. (a) Cells from PTCL were cultured in vitro in the presence or absence of increasing concentrations of cyclosporine A (CsA) (or equal concentrations of excipient, ie ethanol, as control vehicle). Data are shown as mean±SD of quadruplicate determinations for one PTCL and is representative of four experiments performed with two Spn-PTCL and two PBS-PTCL. (b) Mice were transferred with 10⁶ cells from PTCL and left untreated or injected daily by the IP route with 20 mg/Kg of CsA either from day 1 for 2 weeks or from day 21 (i.e. when abdomen enlargement was clinically detectable) for 2 weeks as well. Experiments conducted with three different PTCL originating from either Spn- or PBS-injected p53^{-/-} mice gave similar results. (c) Survival curves of WT mice or CD1d^{-/-} mice transferred with 10⁶ PTCL cells. All mice alive at day 100 were sacrificed and showed absence of macroscopic lymphoma development. Experiments were conducted with four different PTCL from Spn- and PBS-PTCL and gave similar results.

FIG. 3 is a series of graphs showing that blocking CD1d mAbs delays PTCL development. Survival curves of WT mice transferred with 10⁶ PTCL cells and injected twice a week by IP route with 15 mg/kg of either IgG1 control isotype or blocking CD1d mAb either from day 1 (a) or from day 21 (b) (i.e. when abdomen enlargement was clinically detectable) and until mice were considered morbid and euthanized.

FIG. 4 is a series of graphs showing that V81 TCR-expressing human lymphomas are CD1d-restricted PTCL. (a) Flow cytometry analysis showing Vδ1 versus human CD1d tetramer loaded with a-GalCer, sulfatide or unloaded (empty tetramer) on one HSTL patient and histograms of human CD1d-tetramer stainings gated on Vδ1+ lymphoma cells from three HSTL patients. (b) Flow cytometry histograms of human CD1d tetramer stainings gated on Vδ1+, Vδ2+ or TCRαβ+ lymphoma cells from three PTCL-NOS patients. (c) Flow cytometry histograms of human CD1d-tetramer stainings gated on Vδ1+, Vδ2+ or TCRαβ+ leukemic cells from five T-LGL patients.

### DETAILED DESCRIPTION

A first object of the present invention relates to a method for diagnosing a T cell lymphoma as a CD1d-restricted gamma/delta T cell lymphoma in a patient in need thereof comprising i) detecting the presence of CD1d-restricted gamma/delta T lymphoma cells in a cell lymphoma sample obtained from the patient and ii) concluding that the T cell lymphoma is a CD1d-restricted gamma/delta T cell lymphoma when the presence of CD1d-restricted gamma/delta T cells is detected in the sample. In some embodiments, the diagnostic method of the present invention is particularly suitable for diagnosing a peripheral T cell lymphoma (PTCL). In particular, the diagnostic method of the present invention is particularly suitable for diagnosing PTCL-NOS (not-otherwise specified) or hepatosplenic T-cell lymphoma. The term “hepatosplenic T-cell lymphoma” has its general meaning in the art and refers to a systemic neoplasm comprising medium-sized cytotoxic gamma/delta T-cells that show a significant sinusoidal infiltration in the liver, spleen, and bone marrow (Falchook GS, Vega F, Dang N H, Samaniego F, Rodriguez M A, Champlin R E, et al. Hepatosplenic gamma-delta T-cell lymphoma: clinicopathological features and treatment. Ann Oncol. 2009; 20(6)1080-1085.). HSTL is an uncommon form of PTCL which usually arises from y/6 T cells and primarily affects young male adults with a median age of 34 years. The immunophenotypic profile of γ/δ-hepatosplenic lymphoma usually includes positivity for the γ/δTCR/CD3 complex, CD2 and CD7, while CD4, CD5 and CD57 are often negative. The neoplastic cells commonly display a cytotoxic profile associated with the expression of CD16 and CD56 with variable CD8 expression. In some embodiments, the lymphoma sample is a biopsy sample (e.g. spleen, liver or bone marrow sample) or a blood sample obtained from the patient.

As used herein the expression “gamma/delta T cell” or “γ/δ T cell” has its general meaning in the art and refers to a small subset of T cells that possess a distinct T-cell receptor (TCR) on their surface. Actually, the vast majority of T-lymphocytes (95%) express on their surface a TCRα/β (formed by one a and one β chain), while just a small minority of T cells (nearly 5%) expresses a TCRγ/δ. As two variable-region genes are commonly located inside the δ chain locus, the most representative subsets of γ/δ T cells express either a Vδ1 or a Vδ82 receptor (Gertner J, Scotet E, Poupot M, et al. Lymphocytes: gamma delta. eLS; 2007). Such subgroups, further classified according to different immunophenotypic profile and distinct tissue localization, show dissimilar functions. Vδ1, which represent the minority of γ/δ T-cells preferentially reside within the intestine, skin epithelia, uterus and spleen. They are usually CD5⁻, CD28⁻, CD57⁺, express the naive phenotype-related CD45RA antigen and exhibit high levels of chemokine receptors CXCR4 and CCR7. Conversely, Vδ2 are mainly detected in the peripheral blood as circulating lymphocytes. They express CD5, CD28, low levels of CXCR4 and CD45RO and display a cytotoxic and memory phenotype.

As used herein the expression “CD1d-restricted gamma/delta T cell” refers to a subset of gamma/delta T cells that specifically recognize self lipid-based or foreign lipid-based antigens bound to the major histocompatibility complex (MHC) class I homolog CD1d. As used herein, the term “CD1d” refers to a member of the CD1 (cluster of differentiation 1) family of glycoproteins expressed on the surface of various human antigen-presenting cells.

In some embodiments, the diagnostic method of the present invention comprises detecting CD1d-restricted gamma/delta T cells that are characterized by their lack of reactivity to CD1d-α-GalCer. More particularly the method of the present invention comprises detecting CD1d-restricted gamma/delta T cells that present reactivity to sulfatide. Sulfatide is a sulfated form of β-galactosylceramide (β-GalCer).

In some embodiments, the diagnostic method of the present invention comprises detecting CD1d-restricted gamma/delta T cell expressing a Vδ1 receptor. The presence of the CD1d-restricted gamma/delta T cells in the sample may be determined by any well known method in the art.

For example, the detection assay may consist in a first step consisting in isolating the gamma/delta T cells with a set of binding partners specific for some immunophenotypic markers (e.g. Vδ1, Vδ2, CD4, CD8, CD5, CD28, CD57 . . . ). Typically, said partners are antibodies that are labeled with a detectable substance, such as a fluorophore (e.g. fluorescein isothiocyanate (FITC) or phycoerythrin (PE) or Indocyanine (Cy5)). According to the invention, methods of flow cytometry are methods of interest for isolating the gamma/delta T cells present in the sample. Said methods are well known in the art. For example, fluorescence activated cell sorting (FACS) is used.

In a second step, the assay is based on the use of a specifically cognate antigen (e.g. sulfatide) loaded on CD1d tetramers. Accordingly the gamma/delta T cells present in the sample are bringing into contact with said tetramers. Tetramers assays are well known in the art. To produce tetramers, the carboxyl terminus of CD1d associated with the cognate antigen (e.g. sulfatide), and treated so as to form a tetramer complex having bound hereto a suitable reporter molecule, preferably a fluorochrome such as, for example, fluoroscein isothiocyanate (FITC), phycoerythrin, phycocyanin or allophycocyanin. The tetramers produced bind to the T cell receptors (TcRs) on the CD1d restricted gamma/delta T cells. There is no requirement for in vitro T cell activation or expansion. Following binding, and washing of the T cells to remove unbound or non-specifically bound tetramer, the CD1d-restricted gamma/delta T cells binding specifically to the tetramer may be detected (and quantified) by standard flow cytometry methods, such as, for example, using a FACSCalibur Flow cytometer (Becton Dickinson). The tetramers can also be attached to paramagnetic particles or magnetic beads to facilitate removal of non-specifically bound reporter and cell sorting. Such particles are readily available from commercial sources (eg. Beckman Coulter, Inc., San Diego, Calif., USA). Tetramer staining does not kill the labelled cells; therefore cell integrity is maintained for further analysis.

A further aspect of the invention relates to a method for treating a CD1d-restricted gamma/delta T cell lymphoma as diagnosed by the diagnostic method of present invention comprising administering the patient with a therapeutically effective amount of a CD1d antagonist. As used herein, the term “CD1d antagonist” means any molecule that attenuates signal transduction mediated by the binding of the TCR of the CD1d restricted gamma/delta T cells with the CD1d of an antigen presenting cells. In specific examples of the invention, a CD1d antagonist is a molecule that inhibits, reduces, abolishes or otherwise reduces signal transduction through the TCR. Such decrease may result where: (i) the CD1d antagonist of the invention binds either to the TCR or CD1d without triggering signal transduction, to reduce or block inhibitory signal transduction mediated by CD1d; or (iii) the CD1d antagonist inhibits CD1d expression especially by reducing or abolishing expression of the gene encoding for CD1d. In some embodiments, the CD1d antagonist is an antibody or a portion thereof and more particularly an anti-CD1d antibody or a portion thereof.

In some embodiments, the antibody is a monoclonal antibody. In some embodiments, the antibody is a polyclonal antibody. In some embodiments, the antibody is a humanized antibody. In some embodiments, the antibody is a chimeric antibody. In some embodiments, the portion of the antibody comprises a light chain of the antibody. In some embodiments, the portion of the antibody comprises a heavy chain of the antibody. In some embodiments, the portion of the antibody comprises a Fab portion of the antibody. In some embodiments, the portion of the antibody comprises a F(ab′)2 portion of the antibody. In some embodiments, the portion of the antibody comprises a Fc portion of the antibody. In some embodiments, the portion of the antibody comprises a Fv portion of the antibody. In some embodiments, the portion of the antibody comprises a variable domain of the antibody. In some embodiments, the portion of the antibody comprises one or more CDR domains of the antibody.

As used herein, “antibody” includes both naturally occurring and non-naturally occurring antibodies. Specifically, “antibody” includes polyclonal and monoclonal antibodies, and monovalent and divalent fragments thereof. Furthermore, “antibody” includes chimeric antibodies, wholly synthetic antibodies, single chain antibodies, and fragments thereof. The antibody may be a human or nonhuman antibody. A nonhuman antibody may be humanized by recombinant methods to reduce its immunogenicity in man.

Antibodies are prepared according to conventional methodology. Monoclonal antibodies may be generated using the method of Kohler and Milstein (Nature, 256:495, 1975). To prepare monoclonal antibodies useful in the invention, a mouse or other appropriate host animal is immunized at suitable intervals (e.g., twice-weekly, weekly, twice-monthly or monthly) with antigenic forms of CD1d. The animal may be administered a final “boost” of antigen within one week of sacrifice. It is often desirable to use an immunologic adjuvant during immunization. Suitable immunologic adjuvants include Freund's complete adjuvant, Freund's incomplete adjuvant, alum, Ribi adjuvant, Hunter's Titermax, saponin adjuvants such as QS21 or Quil A, or CpG-containing immunostimulatory oligonucleotides. Other suitable adjuvants are well-known in the field. The animals may be immunized by subcutaneous, intraperitoneal, intramuscular, intravenous, intranasal or other routes. A given animal may be immunized with multiple forms of the antigen by multiple routes.

Briefly, the recombinant CD1d may be provided by expression with recombinant cell lines. CD1d may be provided in the form of human cells expressing CD1d at their surface. Recombinant forms of CD1d may be provided using any previously described method. Following the immunization regimen, lymphocytes are isolated from the spleen, lymph node or other organ of the animal and fused with a suitable myeloma cell line using an agent such as polyethylene glycol to form a hydridoma. Following fusion, cells are placed in media permissive for growth of hybridomas but not the fusion partners using standard methods, as described (Coding, Monoclonal Antibodies: Principles and Practice: Production and Application of Monoclonal Antibodies in Cell Biology, Biochemistry and Immunology, 3rd edition, Academic Press, New York, 1996). Following culture of the hybridomas, cell supernatants are analyzed for the presence of antibodies of the desired specificity, i.e., that selectively bind the antigen. Suitable analytical techniques include ELISA, flow cytometry, immunoprecipitation, and western blotting. Other screening techniques are well-known in the field. Preferred techniques are those that confirm binding of antibodies to conformationally intact, natively folded antigen, such as non-denaturing ELISA, flow cytometry, and immunoprecipitation.

Significantly, as is well-known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). The Fc′ and Fc regions, for example, are effectors of the complement cascade but are not involved in antigen binding. An antibody from which the pFc′ region has been enzymatically cleaved, or which has been produced without the pFc′ region, designated an F(ab′)2 fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDRS). The CDRs, and in particular the CDRS regions, and more particularly the heavy chain CDRS, are largely responsible for antibody specificity.

It is now well-established in the art that the non CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody.

This invention provides in certain embodiments compositions and methods that include humanized forms of antibodies. As used herein, “humanized” describes antibodies wherein some, most or all of the amino acids outside the CDR regions are replaced with corresponding amino acids derived from human immunoglobulin molecules. Methods of humanization include, but are not limited to, those described in U.S. Pat. Nos. 4,816,567, 5,225,539, 5,585,089, 5,693,761, 5,693,762 and 5,859,205, which are hereby incorporated by reference. The above U.S. Pat. Nos. 5,585,089 and 5,693,761, and WO 90/07861 also propose four possible criteria which may used in designing the humanized antibodies. The first proposal was that for an acceptor, use a framework from a particular human immunoglobulin that is unusually homologous to the donor immunoglobulin to be humanized, or use a consensus framework from many human antibodies. The second proposal was that if an amino acid in the framework of the human immunoglobulin is unusual and the donor amino acid at that position is typical for human sequences, then the donor amino acid rather than the acceptor may be selected. The third proposal was that in the positions immediately adjacent to the 3 CDRs in the humanized immunoglobulin chain, the donor amino acid rather than the acceptor amino acid may be selected. The fourth proposal was to use the donor amino acid reside at the framework positions at which the amino acid is predicted to have a side chain atom within 3A of the CDRs in a three dimensional model of the antibody and is predicted to be capable of interacting with the CDRs. The above methods are merely illustrative of some of the methods that one skilled in the art could employ to make humanized antibodies. One of ordinary skill in the art will be familiar with other methods for antibody humanization.

In some embodiments of the humanized forms of the antibodies, some, most or all of the amino acids outside the CDR regions have been replaced with amino acids from human immunoglobulin molecules but where some, most or all amino acids within one or more CDR regions are unchanged. Small additions, deletions, insertions, substitutions or modifications of amino acids are permissible as long as they would not abrogate the ability of the antibody to bind a given antigen. Suitable human immunoglobulin molecules would include IgGI, IgG2, IgG3, IgG4, IgA and IgM molecules. A “humanized” antibody retains a similar antigenic specificity as the original antibody. However, using certain methods of humanization, the affinity and/or specificity of binding of the antibody may be increased using methods of “directed evolution”, as described by Wu et al., I. Mol. Biol. 294:151, 1999, the contents of which are incorporated herein by reference.

Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference. These animals have been genetically modified such that there is a functional deletion in the production of endogenous (e.g., murine) antibodies. The animals are further modified to contain all or a portion of the human germ-line immunoglobulin gene locus such that immunization of these animals will result in the production of fully human antibodies to the antigen of interest. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (KAMA) responses when administered to humans.

In vitro methods also exist for producing human antibodies. These include phage display technology (U.S. Pat. Nos. 5,565,332 and 5,573,905) and in vitro stimulation of human B cells (U.S. Pat. Nos. 5,229,275 and 5,567,610). The contents of these patents are incorporated herein by reference.

Thus, as will be apparent to one of ordinary skill in the art, the present invention also provides for F(ab′) 2 Fab, Fv and Fd fragments; chimeric antibodies in which the Fc and/or FR and/or CDR1 and/or CDR2 and/or light chain CDR3 regions have been replaced by homologous human or non-human sequences; chimeric F(ab′)2 fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. The present invention also includes so-called single chain antibodies. The various antibody molecules and fragments may derive from any of the commonly known immunoglobulin classes, including but not limited to IgA, secretory IgA, IgE, IgG and IgM. IgG subclasses are also well known to those in the art and include but are not limited to human IgGI, IgG2, IgG3 and IgG4.

In another embodiment, the antibody according to the invention is a single domain antibody. The term “single domain antibody” (sdAb) or “VHH” refers to the single heavy chain variable domain of antibodies of the type that can be found in Camelid mammals which are naturally devoid of light chains. Such VHH are also called “nanobode”. According to the invention, sdAb can particularly be llama sdAb.

In some embodiments, the CD1d antagonist is an aptamer. Aptamers are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library. The random sequence library is obtainable by combinatorial chemical synthesis of DNA. In this library, each member is a linear oligomer, eventually chemically modified, of a unique sequence. Peptide aptamers consists of a conformationally constrained antibody variable region displayed by a platform protein, such as E. coli Thioredoxin A that are selected from combinatorial libraries by two hybrid methods.

In some embodiments the CD1d antagonist is an inhibitor of CD1d expression. An “inhibitor of expression” refers to a natural or synthetic compound that has a biological effect to inhibit the expression of a gene. Therefore, an “inhibitor of CD1d expression” denotes a natural or synthetic compound that has a biological effect to inhibit the expression of CD1d gene. In a preferred embodiment of the invention, said inhibitor of gene expression is a siRNA, an antisense oligonucleotide or a ribozyme.

Inhibitors of gene expression for use in the present invention may be based on antisense oligonucleotide constructs. Anti-sense oligonucleotides, including anti-sense RNA molecules and anti-sense DNA molecules, would act to directly block the translation of CD1d mRNA by binding thereto and thus preventing protein translation or increasing mRNA degradation, thus decreasing the level of CD1d, and thus activity, in a cell. For example, antisense oligonucleotides of at least about 15 bases and complementary to unique regions of the mRNA transcript sequence encoding CD1d can be synthesized, e.g., by conventional phosphodiester techniques and administered by e.g., intravenous injection or infusion. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732).

Small inhibitory RNAs (siRNAs) can also function as inhibitors of gene expression for use in the present invention. Gene expression can be reduced by contacting the tumor, subject or cell with a small double stranded RNA (dsRNA), or a vector or construct causing the production of a small double stranded RNA, such that gene expression is specifically inhibited (i.e. RNA interference or RNAi). Methods for selecting an appropriate dsRNA or dsRNA-encoding vector are well known in the art for genes whose sequence is known (e.g. see Tuschi, T. et al. (1999); Elbashir, S. M. et al. (2001); Hannon, GJ. (2002); McManus, M T. et al. (2002); Brummelkamp, TR. et al. (2002); U.S. Pat. Nos. 6,573,099 and 6,506,559; and International Patent Publication Nos. WO 01/36646, WO 99/32619, and WO 01/68836).

Ribozymes can also function as inhibitors of gene expression for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of CD1d mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

Both antisense oligonucleotides and ribozymes useful as inhibitors of gene expression can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Antisense oligonucleotides siRNAs and ribozymes of the invention may be delivered in vivo alone or in association with a vector. In its broadest sense, a “vector” is any vehicle capable of facilitating the transfer of the antisense oligonucleotide siRNA or ribozyme nucleic acid to the cells. Preferably, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the the antisense oligonucleotide siRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rouse sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

Preferred viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in KRIEGLER (A Laboratory Manual,” W.H. Freeman C.O., New York, 1990) and in MURRY (“Methods in Molecular Biology,” vol. 7, Humana Press, Inc., Cliffton, N.J., 1991).

Preferred viruses for certain applications are the adeno-viruses and adeno-associated viruses, which are double-stranded DNA viruses that have already been approved for human use in gene therapy. The adeno-associated virus can be engineered to be replication deficient and is capable of infecting a wide range of cell types and species. It further has advantages such as, heat and lipid solvent stability; high transduction frequencies in cells of diverse lineages, including hemopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other vectors include plasmid vectors. Plasmid vectors have been extensively described in the art and are well known to those of skill in the art. See e.g., SANBROOK et al., “Molecular Cloning: A Laboratory Manual,” Second Edition, Cold Spring Harbor Laboratory Press, 1989. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUCl9, pRC/CMV, SV40, and pBlueScript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

By a “therapeutically effective amount” of CD1d antagonist as above described is meant a sufficient amount of the CD1d antagonist that is effective for producing some desired therapeutic effect (e.g. decreasing the survival of CD1d-restricted gamma/delta T lymphoma cells). It will be understood, however, that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidential with the specific polypeptide employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

In some embodiments, the CD1d antagonist is administered to the patient in combination with a calcineurin inhibitor, such as cyclosporine A. The CD1d antagonist of the invention is administered to the patient in the form of a pharmaceutical composition. Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The CD1d antagonist can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active compounds in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the typical methods of preparation are vacuum-drying and freeze-drying techniques which yield a powder of the active ingredient plus any additional desired ingredient from a previously sterile-filtered solution thereof. The preparation of more, or highly concentrated solutions for direct injection is also contemplated, where the use of DMSO as solvent is envisioned to result in extremely rapid penetration, delivering high concentrations of the active agents to a small tumor area. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. Some variation in dosage will necessarily occur depending on the condition of the subject being treated. The person responsible for administration will, in any event, determine the appropriate dose for the individual subject.

### EXAMPLE 1

### CD1d Restricted T Cell-Lymphomas in Humans

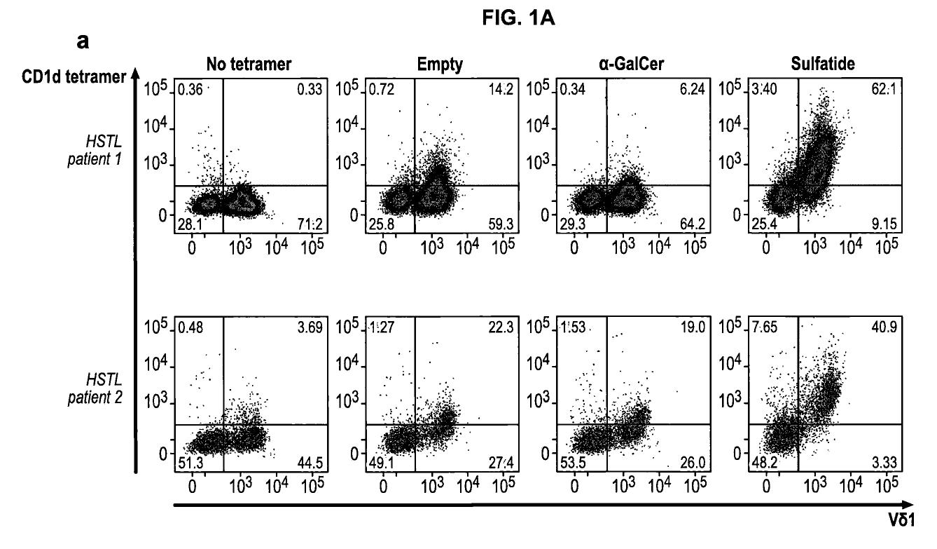

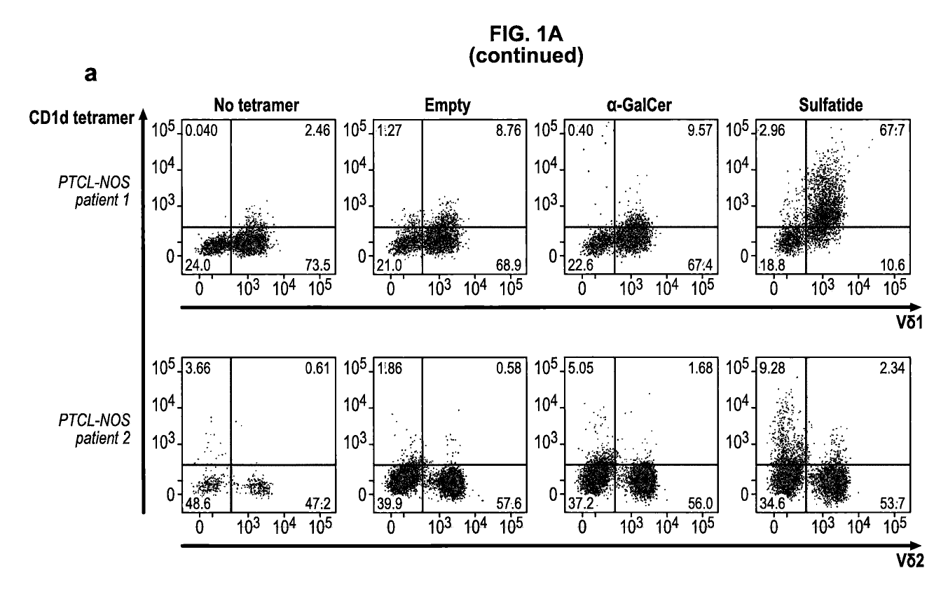

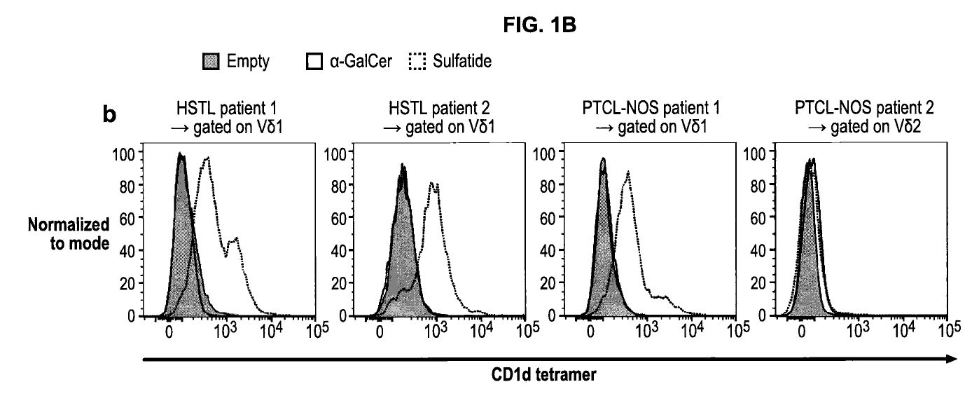

Since V_{β} and, to a lesser extent, V_{α} repertoire in T-cell malignancies have been investigated and no semi-invariant chain usage has been observed in a specific entity, it is unlikely that human peripheral T-cell lymphoma (PTCL) could derive from invariant NKT (iNKT) cells. Furthermore, iNKT are more than 10-fold less abundant in humans than in mice. Although the full extent of CD1d recognition by γδ T cells has yet to be determined, γδ T cells appear to be a substantial complement to NKT cells in the surveillance of lipid antigens (Bendelac et al., 2007). CD1d-mediated lipid-antigen recognition has been described for γδ T cells in both the circulation and the intestinal epithelium (Agea et al., 2005 ; Bai et al., 2012 ; Mangan et al., 2013 ; Russano et al., 2007). These results have been recently extended by the crystallization of Vδ1 TCR in complex with CD1d and αGalCer or the self-lipid sulfatide (Luoma et al., 2013; Uldrich et al., 2013). Hepatosplenic T-cell lymphoma (HSTL) is a PTCL entity mostly derived from γδ T cells and more particularly from Vδ1 T cells. We therefore hypothesized that HSTL could be CD1d-restricted T cell lymphoma. Vδ1-expressing lymphoma cells from two HSTL patients were stained with sulfatide-loaded human CD1d tetramers but not with the αGalCer-loaded ones (FIG. 1). We also demonstrated that a Vδ1 PTCL-NOS (not-otherwise specified) but not a Vδ2 was also CD1d-sulfatide tetramer positive demonstrating their CD1d-restriction (FIG. 1).

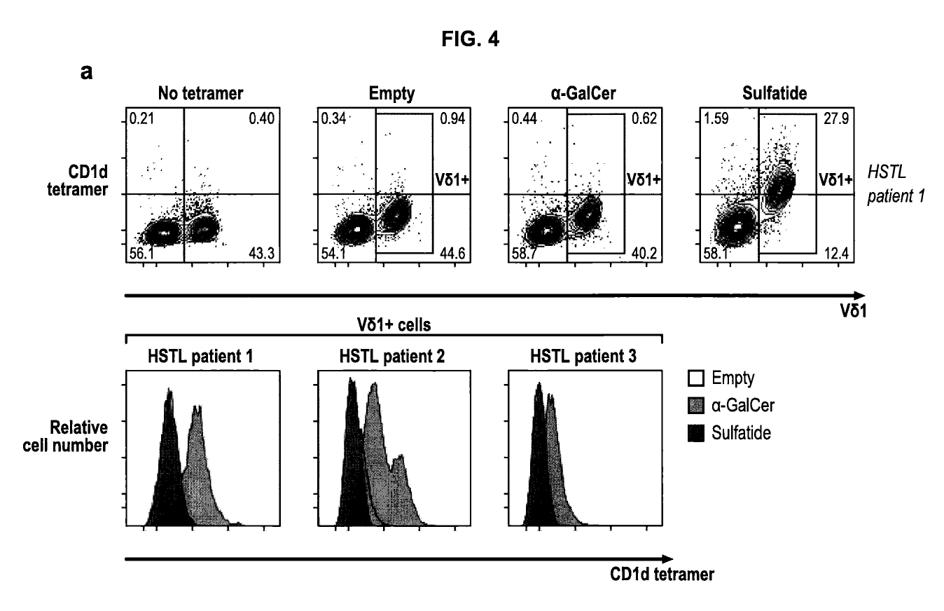

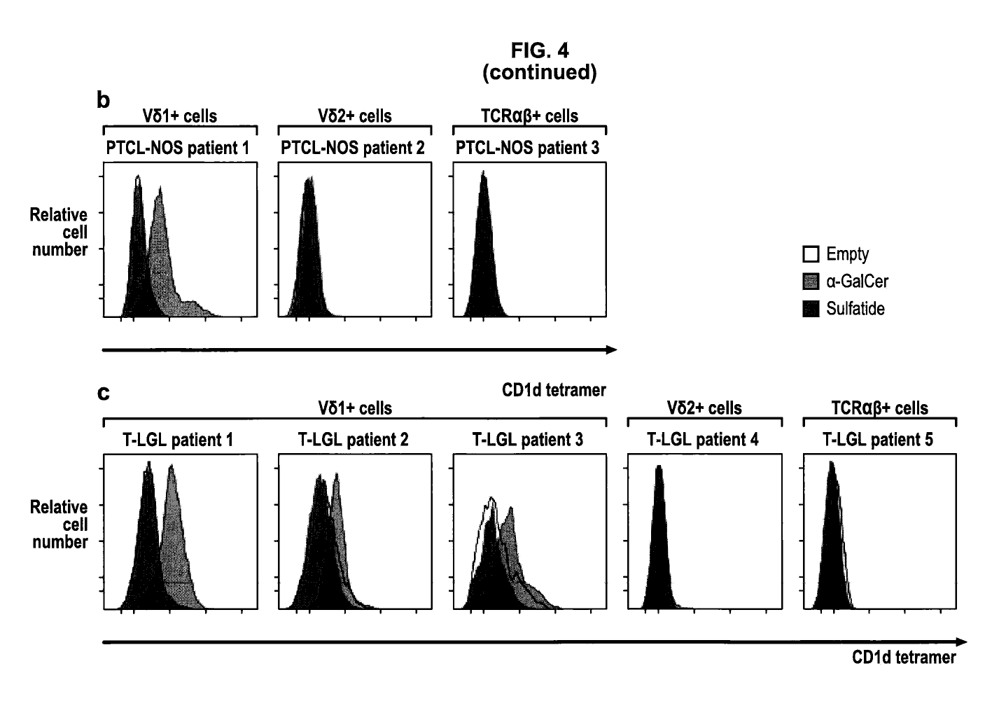

We studied whether HSTL were CD1d-restricted PTCL, using human CD1d-loaded tetramers. In line with our hypothesis, all Vδ1-expressing lymphoma cells from primary HSTL samples specifically bound human CD1d tetramers loaded with sulfatide but not with αGalCer (FIG. 4). To determine whether the CD1d restriction was dictated by the type of PTCL or by the nature of the TCR expressed, we explored the binding of human CD1d tetramers on PTCL-NOS and T-cell large granular lymphocyte leukemia (T-LGL) expressing Vδ1, Vδ2, or αβ TCR. Only T-LGL and PTCL-NOS expressing a Vδ1 TCR, but not Vδ2-expressing and TCRαβ-expressing PTCL-NOS and T-LGL, were also stained by sulfatide-loaded CD1d tetramers confirming a broader CD1d-restriction for all Vδ1-expressing PTCL studied (FIG. 4).

### EXAMPLE 2

### CD1d-Dependent Chronic Stimulation Drives T Cells Lymphomagenesis in Mouse

Methods:

Mice. P53^{-/-} mice (B6.129S2-Trp53^{tm1Tyj}J) were purchased from The Jackson Laboratory. P53^{+/-} mice were generated by crossing p53^{-/-} mice with C57BL/6J WT mice purchased from Charles River Laboratories (I'Arbresle, France). CD3ε^{-/-} mice were obtained from Dr M. Malissen (Centre d'Immunologie Marseille-Luminy, France). CD1d^{-/-} mice were provided by L. Van Kaer (Howard Hughes Medical Institute, Nashville, Tenn.). All these mice were on the C57BI/6J background and were maintained in specific pathogen-free conditions at the Plateau de Biologie Expérimentale de la Souris (Ecole Normale Superieure de Lyon, Fance). All studies and procedures were performed in accordance with EU guidelines and approved by the local Animal Ethics Evaluation Committee (CECCAPP).

S. pneumoniae injections. The encapsulated serotype 3 WU2 strain of S. pneumonia was grown in Todd-Hewitt broth supplemented with 0.5% yeast extract to mid-log phase, then enumerated by plating the suspension on blood agar plates. Bacteria were washed twice in phosphate-buffered saline (PBS) and heat-inactivated by a 1 hours incubation at 60° C. Aliquots of this suspension were stored at −80° C. until use. At the time of injection, 5×10⁶ bacteria were diluted in 200 μL sterile PBS for intraperitoneal injection as previously described⁴². Animal experiments were performed in a biosafety level 2 animal facility.

Flow cytometry analysis. Single-cell suspension prepared from spleen, mesenteric and/or mediastinal lymph nodes, liver and bone marrow were stained with a panel of fluorescent-labeled antibodies. Before staining, Fc receptors were blocked for 15 minutes at 4° C. with 24G2 hybridoma supernatant. The following membrane antibodies (mAbs) reactive with murine cells were purchased from BD Biosciences: CD3c (145-2C11), CD4 (GK1.5), CD8a (53-6.7), B220 (RA3-6B2), CD69 (H1.2F3), CD62L (MEL-14), CD54 (3E2), CD44 (IM7), CD25 (PC61), TCRβ (H57-597), TCRγδ (GL3), CD5 (53-7.3), CD30 (mCD30.1), CD117 (2B8), CD24 (M1/69), CD95 (Jo2), CD103 (M290), CD19 (1D3), CD127 (A7R34), CD122 (TM-β1), Vβ1+5.2 (MR9-34), Vβ8.1+8.2 (MR5-2), Vβ8.3 (1B3.3) and Vδ6.3/2 (8F4H7B7). All antibodies used were used as direct conjugates to FITC, PE, PerCP, PerCP-Cy5.5, APC, PE-Cy7, APC-Cy7, or AlexaFluor-647. PerCP-conjugated anti-Thy1.2 (30-H12) mAb and PE-conjugated anti-V≢51.1 (462.9) reactive with murine cells was purchased from Biolegend. APC-conjugated anti-CCR7 (4612), PE-conjugated anti-PD-1 (J43) and PE-conjugated anti-PLZF (Mags.21F7, anti-mouse and human) were purchased from eBioscience. PE-conjugated CD1d-a-GalCer tetramer (PBS57) was obtained from the NIH Tetramer Core Facility or APC-conjugated CD1d-α-GalCer tetramer (PBS57) from Proimmune. PE-conjugated or APC-conjugated unloaded tetramers from the same supplier were used as control. For all antibodies, corresponding isotype controls were purchased from the same supplier. Data were collected using a LSRII flow cytometer (BD) and analyzed with FlowJo software (Tree Star).

Clonality assessment. Peripheral T-cell lymphoma cells from involved spleen or liver were sorted on a BD FACSAria sorter. Murine T lymphocyte repertoire diversity was measured using Immun'Ig® tests (ImmunID Technologie). Genomic DNA was extracted using standard techniques and Multi-N-plex PCR were performed using an upstream primer specific of all functional members of a given TRBV family and a downstream primer specific of a given TRBJ segment (international ImMunoGeneTics information system, www.imgt.orq). This assay allows the simultaneous exhaustive detection of V-J rearrangements in the same reaction. Each Vx-J1, J2, J3, J4, Jn product was separated as a function of its size and the Constel'ID® software (ImmunID Technologies) was used for further analytical studies including generation of 3D repertoires illustrations.

Tumor transplantation and therapeutic in vivo experiments. If not otherwise specified, 10⁶ freshly isolated whole splenocytes from lymphoma-bearing animals were transferred intravenously by retro-orbital injection into syngeneic immunocompetent (ie C57B1/6J WT animals) or immunocompromised (ie CD3ε^{-/-} mice) mice. For transfers experiment, 10⁶ thawed PTCL from Spn-injected or PBS-injected mice isolated from liver and subsequently cryopreserved in 10% DMSO in liquid nitrogen were injected. PTCL were either transferred in syngeneic CD1d^{-/-} or WT mice. For therapeutic trials, 20 mg/kg of cyclosporin A (Novartis Pharma) or 15 mg/kg of blocking CD1d mAbs (clone HB323; BioXcell) diluted in 200 μL were injected daily or twice a week, respectively, from day 1 following transfer of PTCL or when tumors became clinically apparent in the transplant-recipient mice (ie at day 21). All groups of mice were age- and sex-matched.

Immunostaining. For immunohistochemistry, mouse tissues (thymus, spleen, liver, kidney, lung and mesenteric or mediastinal lymph nodes) were fixed in 10% formaldehyde and paraffin-embedded. Sections of 4-μm thickness were stained for H&E or immunostained with the following antibodies: anti-TdT (A3524, Dako) and anti-Ki67 (SP6, Lab Vision). Sections were viewed using a Leica DMR microscope and images captured with a Nikon Digital Camera, DXM 1200C (Nikon, Tokyo, Japan).

Microarray analysis. Cell sorting. Thymic and peripheral T-cell lymphoma tumoral cells from thymus or liver, respectively, were sorted on a FACSAria sorter (BD Biosciences). Purity was constantly over 98% (not shown). Sorted normal NKT cells from liver and normal T cells pooled from spleen and mesenteric lymph nodes from age-matched WT animal were used as control. For normal T-cell activation, anti-CD3/anti-CD28 coated beads (Invitrogen Dynal AS, Oslo, Norway) were used at a 1:1 bead-to-cell ratio in a 3-days culture.

Target labeling. Total RNA was extracted using TRIZOL Reagent (Invitrogen) and was amplified by two rounds of in vitro transcription (IVT) using ExpressArt® C&E mRNA amplification nano kit (AmpTec GmbH, Hamburg, Germany). During the second IVT amplification RNA was biotin-labeled using BioArray HighYield RNA Transcript Labeling Kit (Enzo Life Sciences, Inc., Farmingdale, N.Y., USA). Before amplification, spikes of synthetic mRNA (GeneChip® Eukaryotic Poly-A RNA Controls, Affymetrix, Santa Clara, Calif., USA) at different concentrations were added to all samples; these positive controls were used to ascertain the quality of the process. Biotinylated antisense cRNA quantification was performed with a Nanodrop 1000 (Nanodrop, Wilmington, Del., USA) and quality checked with Agilent 2100 Bioanalyzer (Agilent technologies, Inc, Palto Alto, Calif., USA).

Arrays hybridization, scanning and normalization. Hybridization was performed following Affymetrix protocol. Briefly, 15 μg of labeled cRNA was fragmented and denaturated in hybridization buffer, then 10 μg was hybridized on GeneChip® Mouse Genome 240 2.0 array (Affymetrix) during 16 hours at 45° C. with constant mixing by rotation at 60 rpm in the Hybridization Oven 640 (Affymetrix). After hybridization, arrays were washed and stained with streptavidin-phycoerythrin (GeneChip® Hybridization Wash and Stain Kit) in the Fluidics Station 450 (Affymetrix) according to the manufacturer's instruction. The arrays were read with a confocal laser (GeneChip® Scanner 3000 7G, Affymetrix). The CEL files were generated using the Affymetrix GeneChip Command Console (AGCC) software 3.0. The obtained data were normalized with Affymetrix Expression Console software using MAS5 statistical algorithm.

IL-7, IL-15 and cyclosporin A in vitro experiments. Lymphoma T cells viability was assessed after 24 hours of culture in medium alone or in medium supplemented with 10 ng/mL of IL-7 (R&D) or IL-15 (R&D) or with increasing concentrations of cyclosporin A (Sigma-Aldrich). Viability was assessed by PTCL staining with FITC-conjugated Annexin V at the indicated time.

M-FISH. Multicolor FISH (M-FISH) technique was performed on chromosome spreads obtained from fixed-cell material and prepared using standard cytogenetic protocols. A mix of 21 labeled painting probes specific for the different mouse chromosomes was used (MetaSystems, Altlussheim, Germany). Experiments were performed according to the manufacturer's protocols. Metaphase spreads were analysed using a fluorescence microscope (Axioplan II ; Zeiss, Oberkochen, Germany) equipped with appropriate filters (DAPI, FITC, Spectrum Orange, TRITC, Cy5 and DEAC). Images were captured and processed using the ISIS/mFISH imaging system (Metasystems).

Statistical analysis. All analyses were performed using GraphPad (GraphPad Software Inc) version 5.0. Histograms represent mean and error bars in all figures represent standard deviation (SD). Comparisons were made with the use of the χ² test or the Fisher's exact test when indicated for categorical variables and with the use of the Mann-Whitney non-parametric test from continuous parameter. Survival curves were constructed with the Kaplan-Meier method and survival distributions were compared by the logrank test. All tests were 2-sided and a p value less than 0.05 was considered as statistically significant.

Results:

Repeated injections of heat-killed streptococcus pneumoniae increase PTCL incidence in p53-deficient mice. p53^{+/-} or p53^{-/-} mice were injected by intraperitoneal (IP) route every 2 weeks either with PBS (control group) or HK-Spn (further on referred to as Spn) until disease development. Three different types of lymphoma were observed based on macroscopic and flow cytometry studies. In agreement with previous reports^{6,7}, the first type was thymic lymphomas (TL) that could be subdivided into localized TL, when only thymus was macroscopically involved, or generalized TL, when tumor cells have spread to liver and spleen. The second was defined as PTCL since no macroscopic thymic involvement was observed as opposed to a massive hepatomegaly and splenomegaly. The third was B-cell lymphoma characterized mainly by enlarged mesenteric lymph nodes and mild liver and spleen enlargement. T-cell vs B-cell determination was established by flow cytometry using anti-CD3 and anti-CD19 staining on single-cell suspensions from involved organs. In p53^{-/-} mice, no difference both in terms of survival or tumor spectrum (solid vs hematopoietic) was observed between the PBS-injected control group and the Spn-injected experimental group. In p53^{+/-} mice, repeated Spn injections shortened survival (P=0.004) primarily by accelerating the development of solid tumors (P=0.007). Strikingly, when considering lymphoma development both in p53^{-/-} and p53^{+/-} mice, PTCL were significantly overrepresented in the Spn-injected group compared to the control group (P=0.03 and P=0.01 respectively). In PTCL, all organs examined (liver, spleen, lung, lymph nodes, bone marrow and kidney) were characterized by a polymorphic infiltrate of predominantly large cells. Architecture was nodular and diffuse with massive infiltration leading to the effacement of the normal structure. Negative TdT staining was observed across all PTCL samples analyzed and whatever the organ (thymus, spleen, liver, lymph node, lung and kidney) as opposed to a strong positive staining in all TL examined, confirming the post-thymic (ie mature) origin of PTCL. We demonstrated clonality of the PTCL using assessment of the TRBV (variable region of the TCR β chain) locus by genomic multiplex PCR and their transferability in both immunodeficient and immunocompetent mice (ie CD3ε KO or C57BI/6 wild type mice respectively).

PTCL are phenotypically and molecularly different from TL. As shown in Table 1, clonality studies of PTCL revealed a striking Vβ repertoire usage bias. Among 13 PTCL tested for Vβ expression as assessed by surface staining or RT-PCR, only Vβ8 and Vβ7 were found to be expressed. Staining with a CD1d-a-galactosylceramide (α-GalCer) tetramer showed that 8 of 8 PTCL tested were positive. Molecular analyses of the TCR Vα chain repertoire demonstrated the expression of the Vα14Jα18 chain in all PTCL tested, confirming their iNKT nature. All three PTCL expressing a γδ TCR were Vγ1.1 and Vδ6.3, in agreement with the previously described population of γδ NKT cells¹⁴.

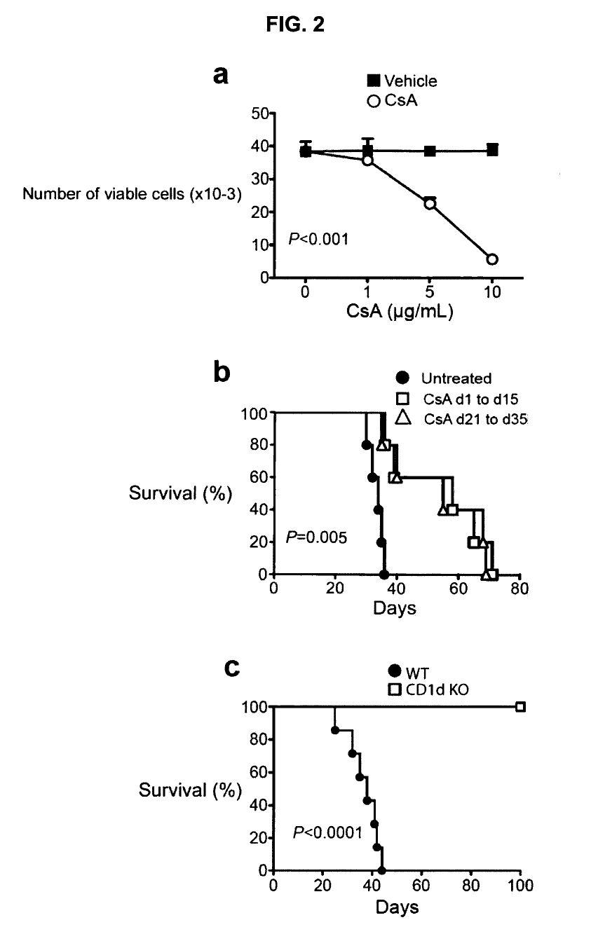

PTCL have molecular features of chronically stimulated T cells and their survival depends on TCR/CD1d interactions. As a first approach to investigate the role of TCR signaling in PTCL survival, we used cyclosporine A (CsA), a calcineurin inhibitor known to strongly suppress TCR signaling. In vitro, increasing concentration of CsA reduced viability of PTCL (FIG. 2a). In vivo, 10⁶ PTCL cells were transferred into C57BU6 syngeneic recipients and CsA was administered daily for 14 days either from day 1 (at day of PTCL transfer) or from day 21 (at appearance of the first clinical signs of lymphoma engraftment) until animals had to be sacrificed. Administration of CsA in both conditions significantly delayed the development of PTCL and increased survival of recipient mice (FIG. 2b), suggesting that PTCL survival relies on the TCR pathway activation for survival. Like memory T cells generated after acute infection, normal NKT cells persist in vivo in the absence of CD1d¹⁶. In keeping with our previous results with CsA and with data demonstrating that chronically stimulated T cells survival relies on TCR engagement by persistent cognate antigens²², PTCL failed to engraft in CD1d^{-/-} mice (FIG. 2c). Altogether these findings demonstrate that CD1d/TCR interactions are required for PTCL survival in vivo.

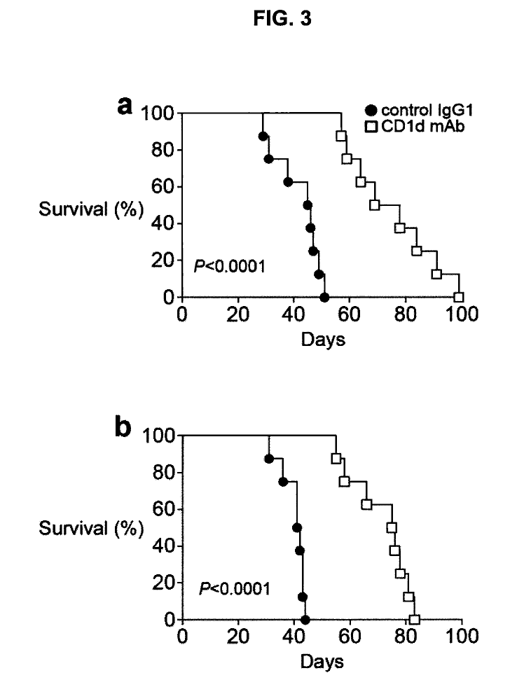

Blocking anti-CD1d antibodies delay PTCL engraftment and prolong survival of recipient mice. Having established that interrupting the CD1d/TCR interaction is detrimental to PTCL survival, we next evaluated the therapeutic potential of in vivo administration of blocking CD1d mAb. PTCL cells were transferred into C57BL/6 syngeneic recipients and blocking CD1d mAb were administered at 15 mg/kg twice a week from day 1 (at day of PTCL transfer) or day 21 (at appearance of the first clinical signs of lymphoma engraftment) until animals had to be sacrificed. Treatment of PTCL-bearing mice with blocking CD1d mAb, but not its IgG1 isotype control, delayed PTCL development and significantly increased mice survival by extending median survival from day 45.5 to 73.5 (P<0.0001) when CD1d mAb were injected from day 1 (FIG. 3a) or from day 41.5 to 75.5 (P<0.0001) when injected from day 21 (FIG. 3b).

Conclusion:

In the present study, we demonstrate for the first time that PTCL that are CD1d restricted can develop in mice. Indeed, repeated injections of heat-killed Spn significantly promoted the development of PTCL both in p53^{+/-} and p53^{-/-} mice. Unlike well described CD4⁺CD8⁺ double positive thymic T-cell lymphomas developing in p53-deficient mice, PTCL exhibited a normal or minute sized thymus, a widespread involvement of all analyzed organs (bone marrow, lung, kidney, spleen and liver) and a lack of immature markers (TdT). Altogether, these findings lead us to consider these lymphomas as of post-thymic origin. Of note, a mild involvement by these peripheral lymphoma cells was found in the thymus without any enlargement, which is in line with the widespread involvement of virtually all organs and possibly the known capacity of mature T cells to re-enter the thymus²⁵. Liver was predominantly involved in all diseased mice. Several lines of evidence demonstrated a contribution of chronic TCR engagement to the development of these PTCL. Actually, cyclosporin A, a TCR signaling inhibitor, decreased cell survival in vitro, and prolonged mice survival following transfer of lymphoma cells into recipient mice. Moreover engraftments of PTCL in mice treated with blocking CD1d mAb or in CD1d^{-/-} mice were partially or completely inhibited compared to engraftments conducted in WT mice. We conclude that PTCL cells displayed a poor engraftment in these conditions because they rely on specific CD1d/TCR interactions to survive and/or to proliferate. The results are in compliance with some observations realized in humans: a link between chronic stimulation by pathogens or self-antigens and lymphomagenesis has been speculated for various types of T cell lymphomas: enteropathy-associated T-cell lymphoma in patients suffering from coeliac disease³³, T-cell large granular lymphocyte leukemia³⁴, hepatosplenic γδ T-cell lymphoma³⁵ and mycosis fungoides ³⁶ or its leukemic variant called Sézary syndrome. Therefore, our present findings that blocking CD1d mAbs impair the development of PTCL in mice would pave the way for the development of a new therapeutic approach for certain PTCL in humans, in particular the CD1d-restricted gamma/delta T cell lymphomas as identified in EXAMPLE 1 of the present specification.

TABLE 1
Characteristics of PTCL in p53-deficient mice.

| | | | | | | | CD1d- | |
|---|---|---|---|---|---|---|---|---|
| | | p53 | | Vα/Vγ | Vβ/Vδ | | tetramer | |
| # | Injections | genotype | TCR | chain^{∞} | chain^{∞} | Coreceptor | binding^{¶} | PLZF^{¶} |
| 1 | PBS | −/− | αβ | Vα14 | Vβ8.2 | DN | + | + |
| 2 | PBS | −/− | αβ | Vα14 | Vβ7 | DN | + | + |
| 3 | PBS | −/− | αβ | Vα14 | Vβ7 | DN | n.d. | n.d. |
| 4 | PBS | −/− | αβ | Vα14 | Vβ7 | DN | + | + |
| 5 | PBS | −/− | αβ | Vα14 | Vβ7 | CD8^{lo} | n.d. | n.d. |
| 6 | PBS | +/− | αβ | n.d. | n.d. | CD4^{lo} | n.d. | n.d. |
| 7 | Spn | −/− | αβ | Vα14 | Vβ8.2 | CD8^{lo} | + | + |
| 8 | Spn | −/− | αβ | Vα14 | Vβ8.3 | DN | + | + |
| 9 | Spn | −/− | αβ | n.d. | Vβ7 | DN | n.d. | n.d. |
| 10 | Spn | −/− | αβ | Vα14 | Vβ8.2 | CD8^{lo} | n.d. | u.d. |
| 11 | Spn | −/− | αβ | Vα14 | Vβ8.2 | CD8^{lo} | n.d. | n.d. |
| 12 | Spn | −/− | αβ | Vα14 | Vβ8.2 | CD8^{lo} | + | + |
| 13 | Spn | +/− | αβ | n.d. | n.d. | DN | n.d. | n.d. |
| 14 | Spn | −/− | αβ | Vα14 | Vβ8 | CD4 | + | + |
| 15 | Spn | +/− | αβ | Vα14 | Vβ8.3 | DN | + | + |
| 16 | Spn | +/− | αβ | n.d. | n.d. | CD4 | n.d. | n.d. |
| 17 | Spn | −/− | γδ | Vγ1.1 | Vδ 6.3 | DN | − | + |
| 18 | Spn | −/− | γδ | Vγ1.1 | Vδ6.3 | DN | − | + |
| 19 | Spn | +/− | γδ | Vγ1.1 | Vδ6.3 | DN | n.d. | n.d. |

^{∞}Vα/Vγ and Vβ/Vδ chains were determined using either antibody staining and flow cyometry analysis or RT-PCR:
^{¶}Assessed by flow cytometry analysis. after fixation and permeabilization for PLZF intracellular staining:
Abbreviations: n.d.. not determined: DN. double negative.

### REFERENCES:

Throughout this application, various references describe the state of the art to which this invention pertains. The disclosures of these references are hereby incorporated by reference into the present disclosure.

Agea, E., Russano, A., Bistoni, O., Mannucci, R., Nicoletti, I., Corazzi, L., Postle, A. D., De Libero, G., Porcelli, S. A., and Spinozzi, F. (2005). Human CD1-restricted T cell recognition of lipids from pollens. J Exp Med 202, 295-308.

Bai, L., Picard, D., Anderson, B., Chaudhary, V., Luoma, A., Jabri, B., Adams, E. J., Savage, P. B., and Bendelac, A. (2012). The majority of CD1d-sulfatide-specific T cells in human blood use a semiinvariant Vdeltal TCR. Eur J Immunol 42, 2505-2510.

Bendelac, A., Savage, P. B., an Teyton, L. (2007). The biology of NKT cells. Annu Rev Immunol 25, 297-336.

Luoma, A. M., Castro, C. D., Mayassi, T., Bembinster, L. A., Bai, L., Picard, D., Anderson, B., Scharf, L., Kung, J. E., Sibener, L. V., et al. (2013). Crystal structure of Vdeltal T cell receptor in complex with CD1d-sulfatide shows MHC-like recognition of a self-lipid by human gammadelta T cells. Immunity 39, 1032-1042.

Mangan, B. A., Dunne, M. R., O'Reilly, V. P., Dunne, P. J., Exley, M. A., O'Shea, D., Scotet, E., Hogan, A. E., and Doherty, D. G. (2013). Cutting edge: CD1d restriction and Th1/Th2/Th17 cytokine secretion by human Vdelta3 T cells. J Immunol 191, 30-34.

Russano, A.M., Bassotti, G., Agea, E., Bistoni, O., Mazzocchi, A., Morelli, A., Porcelli, S.A., and Spinozzi, F. (2007). CD1-restricted recognition of exogenous and self-lipid antigens by duodenal gammadelta+T lymphocytes. J Immunol 178, 3620-3626.

Uldrich, A. P., Le Nours, J., Pellicci, D. G., Gherardin, N. A., McPherson, K. G., Lim, R. T., Patel, O., Beddoe, T., Gras, S., Rossjohn, J., et al. (2013). CD1d-lipid antigen recognition by the gammadelta TCR. Nat Immunol 14, 1137-1145.

1 Suarez, F., Lortholary, O., Hermine, O. & Lecuit, M. Infection-associated lymphomas derived from marginal zone B cells: a model of antigen-driven lymphoproliferation. Blood 107, 3034-3044 (2006).

2 Ghiotto, F. et al. Remarkably similar antigen receptors among a subset of patients with chronic lymphocytic leukemia. J Clin Invest 113, 1008-1016 (2004).

3 Landgren, O. et al. Respiratory tract infections and subsequent risk of chronic lymphocytic leukemia. Blood 109, 2198-2201 (2007).

4 Enno, A., O'Rourke, J., Braye, S., Howlett, R. & Lee, A. Antigen-dependent progression of mucosa-associated lymphoid tissue (MALT)-type lymphoma in the stomach. Effects of antimicrobial therapy on gastric MALT lymphoma in mice. Am J Pathol 152, 1625-1632 (1998).

5 Enno, A. et aL MALToma-like lesions in the murine gastric mucosa after long-term infection with Helicobacter felis. A mouse model of Helicobacter pylori-induced gastric lymphoma. Am J Pathol 147, 217-222 (1995).

6 Donehower, L. A. et al. Mice deficient for p53 are developmentally normal but susceptible to spontaneous tumours. Nature 356, 215-221 (1992).

7 Jacks, T. et aL Tumor spectrum analysis in p53-mutant mice. Curr Biol 4, 1-7 (1994).

8 Harvey, M. et al. Spontaneous and carcinogen-induced tumorigenesis in p53-deficient mice. Nat Genet 5, 225-229 (1993).

9 Tennant, R. W., French, J. E. & Spalding, J. W. Identifying chemical carcinogens and assessing potential risk in short-term bioassays using transgenic mouse models. Environ Health Perspect 103, 942-950 (1995).

10 Ward, J. M. et al. Splenic marginal zone B-cell and thymic T-cell lymphomas in p53-deficient mice. Lab. Invest. 79, 3-14 (1999).

11 Renno, T. et al. Expression of B220 on activated T cell blasts precedes apoptosis. European journal of immunology 28, 540-547 (1998).

12 Liu, G. et al. Chromosome stability, in the absence of apoptosis, is critical for suppression of tumorigenesis in Trp53 mutant mice. Nat Genet 36, 63-68 (2004).

13 Godfrey, D. I., MacDonald, H. R., Kronenberg, M., Smyth, M. J. & Van Kaer, L. NKT cells: what's in a name? Nature reviews. Immunology 4, 231-237 (2004).

14 Lees, R. K., Ferrero, I. & MacDonald, H. R. Tissue-specific segregation of TCRgamma delta+NKT cells according to phenotype TCR repertoire and activation status: parallels with TCR alphabeta+NKT cells. European journal of immunology 31, 2901-2909 (2001).

15 Coquet, J. M. et al. Diverse cytokine production by NKT cell subsets and identification of an IL-17-producing CD4-NK1.1-NKT cell population. Proc Natl Acad Sci USA 105, 11287-11292 (2008).

16 Matsuda, J. L. et al. Homeostasis of V alpha 14i NKT cells. Nat Immunol 3, 966-974 (2002).

17 Wilson, M. T. et al. The response of natural killer T cells to glycolipid antigens is characterized by surface receptor down-modulation and expansion. Proceedings of the National Academy of Sciences of the United States of America 100, 10913-10918 (2003).

18 Chang, W. S. et al. Cutting edge: Programmed death-1/programmed death ligand 1 interaction regulates the induction and maintenance of invariant NKT cell anergy. Journal of immunology 181, 6707-6710 (2008).

19 Subramanian, A. et al. Gene set enrichment analysis: a knowledge-based approach for interpreting genome-wide expression profiles. Proceedings of the National Academy of Sciences of the United States of America 102, 15545-15550, doi:10.1073/pnas.0506580102 (2005).

20 Liberzon, A. et al. Molecular signatures database (MSigDB) 3.0. Bioinformatics 27, 1739-1740 (2011).

21 Baniyash, M. TCR zeta-chain downregulation: curtailing an excessive inflammatory immune response. Nature reviews. Immunology 4, 675-687 (2004).

22 Shin, H., Blackburn, S. D., Blattman, J. N. & Wherry, E. J. Viral antigen and extensive division maintain virus-specific CD8 T cells during chronic infection. The Journal of experimental medicine 204, 941-949 (2007).

23 Swerdlow, S., Campo, E. & Harris, N. World Health Organization Classification of Tumors of Haematopoietic and Lymphoid Tissues. IARC Press (2008).

24 de Leval, L. et al. The gene expression profile of nodal peripheral T-cell lymphoma demonstrates a molecular link between angioimmunoblastic T-cell lymphoma (AITL) and follicular helper T (TFH) cells. Blood 109, 4952-4963 (2007).

25 Sprent, J. & Surh, C. D. Re-entry of mature T cells to the thymus: an epiphenomenon? Immunology and cell biology 87, 46-49 (2009).

26 Wherry, E. J. T cell exhaustion. Nature immunology 12, 492-499 (2011).

27 Kinjo, Y. et al. Invariant natural killer T cells recognize glycolipids from pathogenic Gram-positive bacteria. Nat Immunol 12, 966-974 (2011).

28 Kawakami, K. et al. Critical role of Valpha14+ natural killer T cells in the innate phase of host protection against Streptococcus pneumoniae infection. Eur J Immunol 33, 3322-3330 (2003).

29 Brigl, M. et al. Innate and cytokine-driven signals, rather than microbial antigens, dominate in natural killer T cell activation during microbial infection. J Exp Med 208, 1163-1177 (2011).

30 Kuppers, R. Mechanisms of B-cell lymphoma pathogenesis. Nat Rev Cancer 5, 251-262 (2005).

31 Pechloff, K. et al. The fusion kinase ITK-SYK mimics a T cell receptor signal and drives oncogenesis in conditional mouse models of peripheral T cell lymphoma. Journal of Experimental Medicine 207, 1031-1044 (2010).

32 Wang, X. et aL TCR-dependent transformation of mature memory phenotype T cells in mice. The Journal of clinical investigation 121, 3834-3845 (2011).

33 Isaacson, P. G. & Du, M. Q. Gastrointestinal lymphoma: where morphology meets molecular biology. J Pathol 205, 255-274 (2005).

34 Rodriguez-Caballero, A. et al. Expanded cells in monoclonal TCR-alphabeta+/CD4+/N Ka+/CD8−/+dim T-LGL lymphocytosis recognize hCMV antigens. Blood 112, 4609-4616(2008).

35 Belhadj, K. Hepatosplenic T-cell lymphoma is a rare clinicopathologic entity with poor outcome: report on a series of 21 patients. Blood 102, 4261-4269 (2003).

36 Tothova, S. M., Bonin, S., Trevisan, G. & Stanta, G. Mycosis fungoides: is it a Borrelia burgdorferi-associated disease? British journal of cancer 94, 879-883 (2006).

37 Jahng, A. et al. Prevention of autoimmunity by targeting a distinct, noninvariant CD1d-reactive T cell population reactive to sulfatide. The Journal of experimental medicine 199, 947-957 (2004).

38 Exley, M. A. et al. A major fraction of human bone marrow lymphocytes are Th2-like CD1d-reactive T cells that can suppress mixed lymphocyte responses. J Immunol 167, 5531-5534 (2001).

39 Murray, A., Cuevas, E. C., Jones, D. B. & Wright, D. H. Study of the immunohistochemistry and T cell clonality of enteropathy-associated T cell lymphoma. AJPA 146, 509-519 (1995).

40 Sturm, A., Itoh, J., Jacobberger, J. W. & Fiocchi, C. p53 negatively regulates intestinal immunity by delaying mucosal T cell cycling. The Journal of clinical investigation 109, 1481-1492 (2002).

41 Qi, Q. et al. A unique role for ITK in survival of invariant NKT cells associated with the p53-dependent pathway in mice. Journal of immunology 188, 3611-3619 (2012).

1-8. (cancelled)

9. A method for treating a CD1d restricted gamma/delta T cell lymphoma in a subject in need thereof, the method comprising,
administering a therapeutically effective amount of a CD1d antagonist to the subject.

10. The method of claim 9, wherein the CD1d antagonist is an anti-CD1d antibody or a portion thereof.

11. The method of claim 10, wherein the anti-CD1d antibody is a monoclonal antibody.

12. The method of claim 10, wherein the anti-CD1d antibody is a humanized antibody, a chimeric antibody, a human antibody, or a single domain antibody.

13. The method of claim 9, wherein the CD1d antagonist is an aptamer.

14. The method of claim 9, wherein the CD1d antagonist is an inhibitor of CD1d expression.

15. The method of claim 9, wherein the CD1d antagonist is administered to the patient in combination with a calcineurin inhibitor.

16. The method of claim 9, wherein the calcinueurin inhibitor is cyclosporine A.

17. The method of claim 9, wherein, prior to the administering, the subject is diagnosed as having a CD1d-restricted γ/δ T cell lymphoma by a method comprising:
isolating γ/δ T cells from a T cell lymphoma sample taken from the subject;
detecting T cells with the sulfatide loaded onto CD1d; and
diagnosing the subject with CD1d-restricted γ/δ T cell lymphoma when binding of the a-galactosylceramide loaded onto CD1d with the γ/δ T cells is not detected or binding of the sulfatide loaded onto CD1d with the γ/δ T cells is detected.

18. A method of detecting CD1d-restricted γ/δ T cell lymphoma in a subject with a T cell lymphoma, the method comprising:
isolating γ/δ T cells from a T cell lymphoma sample taken from the subject;
detecting whether CD1d-restricted γ/δ T cells are present in the T cell lymphoma sample by either:
contacting the γ/δ T cells with a-galactosylceramide loaded onto CD1d and detecting binding between the γ/δ T cells with the a-galactosylceramide loaded onto CD1d, or
contacting the γ/δ T cells with sulfatide loaded onto CD1d and detecting binding between the γ/δ T cells with the sulfatide loaded onto CD1d; and
diagnosing the subject with CD1d-restricted γ/δ T cell lymphoma when binding of the a-galactosylceramide loaded onto CD1d with the γ/δ T cells is not detected or binding of the sulfatide loaded onto CD1d with the γ/δ T cells is detected.

19. The method of claim 18, wherein the T cell lymphoma is a peripheral T cell lymphoma (PTCL).

20. The method of claim 19, wherein the PTCL is a PTCL-not-otherwise specified (PTCL-NOS) or a hepatosplenic T-cell lymphoma (HSTL).

21. The method of claim 18, wherein the T cell lymphoma sample is a biopsy sample or a blood sample obtained from the subject.

22. The method of claim 21, wherein the biopsy sample is selected from the group consisting of a spleen sample, a liver sample, and a bone marrow sample.

23. The method of claim 18, wherein the CDd1 of the a-galactosylceramide loaded onto CD1d or the CDd1 of the sulfatide loaded onto CD1d is a CD1d tetramer.

24. The method of claim 18, further comprising:
detecting a Vδ1 receptor on the γ/δ T cells and diagnosing the subject with CD1d-restricted γ/δ T cell lymphoma when the Vδ1 receptor is detected.

25. A method of diagnosing and treating CD1d-restricted γ/δ T cell lymphoma in a subject, the method comprising:
diagnosing the subject with CD1d-restricted γ/δ T cell lymphoma according to the method of claim 18; and
administering a therapeutically effective amount of a CD1d antagonist to the subject, wherein the CD1d antagonist is an anti-CD1d antibody, a portion of an anti-CD1d antibody, siRNA that blocks expression of CD1d, an antisense oligonucleotide that blocks expression of CD1d, or a ribozyme that blocks expression of CD1d.

26. A method of detecting CD1d-restricted γ/δ T cell lymphoma in a subject with a T cell lymphoma, the method comprising:
isolating γ/δ T cells from the T cell lymphoma sample taken from the subject; and
detecting whether CD1d-restricted γ/δ T cells are present in the T cell lymphoma sample by at least one of:
contacting the γ/δ T cells with a first tetramer complex comprising a-galactosylceramide loaded onto a first CD1d tetramer and a first reporter molecule; washing the γ/δ T cells to remove unbound or non-specifically bound first tetramer complex; and unsuccessfully detecting the first reporter molecule on the washed γ/δ T cells, or
contacting the γ/δ T cells with a second tetramer complex comprising sulfatide loaded onto a second CD1d tetramer and a second reporter molecule; washing the γ/δ T cells to remove unbound or non-specifically bound second tetramer complex; and detecting the second reporter molecule on the washed γ/δ T cells.

27. The method of claim 26, further comprising:
administering a therapeutically effective amount of a CD1d antagonist to the subject when CD1d-restricted γ/δ T cells are present in the T cell lymphoma sample.

28. The method of claim 26, wherein the isolating γ/δ T cells from the T cell lymphoma sample comprises:
contacting the T cell lymphoma sample with antibodies directed against an immunophenotypic marker expressed by γ/δ T cells selected from the group consisting of Vδ1, Vδ2, CD4, CD8, CD54, CD28, and CD57, wherein the antibodies are labelled with a fluorophore; and
isolating γ/δ T cells from the T cell lymphoma by flow cytometry.

29. The method of claim 26, wherein the first reporter molecule and the second reporter molecule are individually selected from the group consisting of a fluorochrome, paramagnetic beads, and magnetic beads.

30. The method of claim 26, wherein the method does not comprise in vitro T cell activation or expansion.
